(19) United States
(12) Patent Application Publication
(10) Pub. No.: US 2023/0122558 A1
(43) Pub. Date: Apr. 20, 2023

(54) METHODS AND SYSTEMS FOR ORTHODONTIC TREATMENT PLANNING WITH VIRTUAL JAW ARTICULATOR

(71) Applicant: CVSTOM Co., San Francisco, CA (US)

(72) Inventor: Kjeld A. AAMODT, San Francisco, CA (US)

(21) Appl. No.: 17/938,520

(22) Filed: Oct. 6, 2022

Related U.S. Application Data
(60) Provisional application No. 63/256,880, filed on Oct. 18, 2021.

(51) Int. Cl. A61C 7/00; A61C 7/08; A61C 9/00; A61C 11/00

(52) U.S. Cl. CPC A61C 7/002; A61C 7/08; A61C 9/0046; A61C 11/00

(57) ABSTRACT

A method of orthodontic treatment planning for a patient includes receiving three-dimensional intraoral surface scan data of a dentition of the patient, receiving three-dimensional volumetric scan data of a dentition of the patient, and determining, for use in planning an orthodontic treatment, a mandibular rotation axis and a glenoid fossae of the temporal bone based on a mandibular condyle of the patient using the scan data.

### CROSS-REFERENCE TO RELATED APPLICATIONS

This application claims priority to U.S. Patent Application Ser. No. 63/256,880 filed Oct. 18, 2021, which is hereby incorporated in its entirety by this reference.

### TECHNICAL FIELD

This invention relates generally to the field of orthodontic treatment planning.

### BACKGROUND

Orthodontia is a specialty of dentistry that aims to correct a patient's teeth and jaws that are improperly positioned, such as for health and/or cosmetic reasons. Generally, orthodontic treatments leverage the application of external forces to cause the progressive movement of one or more teeth from their original improper positions to desired positions. Some conventional orthodontic treatments involve bonding brackets to tooth surfaces and progressively adjusting wires coupled to the brackets, in order to urge teeth toward desired positions and orientations. Another conventional orthodontic treatment involves the wearing of clear aligner trays with tooth-receiving cavities, to progressively move teeth toward final positions and orientations over a predetermined period of time.

Planning such orthodontic treatments includes obtaining a physical model and/or digital model of a patient's dentition and using such models to generate proposed treatment paths for each tooth to be moved. For example, a physical model of a patient's dentition can be obtained through a mold impression, while a digital model can be obtained by scanning the patient's dentition (and/or a physical model of the patient's dentition) with a scanning device. However, such modeling methods are limited in the amount of patient information that may be obtained, thereby leading to inaccurate treatment plans and longer total treatment times. Thus, there is a need for improved methods and systems for orthodontic treatment planning.

### SUMMARY

Generally, a method of orthodontic treatment planning for a patient includes receiving three-dimensional volumetric scan data of a dentition of the patient, and determining, for use in planning an orthodontic treatment, a mandibular rotation axis and a glenoid fossae of the temporal bone based on a mandibular condyle of the patient using the scan data.

In some variations, three-dimensional intraoral surface scan data of the dentition may be received, and the intraoral surface scan data and the volumetric scan data may be overlayed to generate integrated scan data. In some variations, overlaying the intraoral surface scan data and the volumetric scan data may comprise registering the intraoral surface scan data with the volumetric scan data.

In some variations, the volumetric scan data may comprise one or more of X-ray scan data and magnetic resonance imaging scan data. In some variations, the volumetric scan data may correspond to a cranium and viscerocranium of the patient. In some variations, the intraoral surface scan data may comprise optical color scan data.

In some variations, the mandibular rotation axis and the glenoid fossae may be based on a lateral, medial, superior, and anterior geometry of the mandibular condyle. In some variations, the mandibular rotation axis and the glenoid fossae may be based on the anterio-superior-most portion of the mandibular condyle. In some variations, the mandibular condyle may be predicted using the scan data input to a machine learning model.

In some variations, a jaw model of the patient may be generated based on the scan data, the mandibular rotation axis, and the glenoid fossae. In some variations, a jaw movement of the patient may be predicted using the jaw model of the patient. In some variations, predicting the jaw movement may comprise one or more of a mandibular movement, articular movement, and movement at occlusion of teeth. In some variations, the mandibular movement may comprise one or more of a hinge, a protrusion, and a lateral movement. In some variations, the articular movement may comprise one or more of a rotation, a translation, a protrusive condylar path, a progressive condylar path, a laterotrusive condylar path, mediotrusive condylar path, a condylar path angle, a Bennett angle, and a Bennett movement. In some variations, the movement at occlusion of teeth may comprise one or more of an incisal path, an incisal path angle, an incisal path distance, an occlusal guidance, and an occlusal interference. In some variations, the orthodontic treatment may be planned using the jaw model of the patient.

In some variations, a plurality of aligner trays may be generated with tooth-receiving cavities, each aligner tray corresponding to a respective tooth arrangement.

Generally, a system for orthodontic treatment planning for a patient includes at least one memory device configured to receive and store three-dimensional volumetric scan data of the dentition, and at least one processor configured to determine, for use in planning an orthodontic treatment, a mandibular rotation axis and a glenoid fossae of the temporal bone based on a mandibular condyle of the patient using the scan data.

In some variations, at least one processor may be configured to receive three-dimensional intraoral surface scan data of the dentition, and overlay the intraoral surface scan data and the volumetric scan data to generate integrated scan data. In some variations, overlaying the intraoral surface scan data and the volumetric scan data may comprise registering the intraoral surface scan data with the volumetric scan data.

In some variations, the volumetric scan data may comprise one or more of X-ray scan data and magnetic resonance imaging scan data. In some variations, the volumetric scan data may correspond to a cranium and viscerocranium of the patient. In some variations, the intraoral surface scan data may comprise optical color scan data.

In some variations, the mandibular rotation axis and the glenoid fossae may be based on a lateral, medial, superior, and anterior geometry of the mandibular condyle. In some variations, the mandibular rotation axis and the glenoid fossae may be based on the anterio-superior-most portion of the mandibular condyle.

In some variations, the at least one processor may be configured to predict the mandibular condyle using the scan data input to a machine learning model. In some variations, the at least one processor may be configured to generate a jaw model of the patient based on the scan data, the mandibular rotation axis, and the glenoid fossae. In some variations, the at least one processor may be configured to predict a jaw movement of the patient using the jaw model of the patient. In some variations, the at least one processor configured to predict the jaw movement may comprise one or more of a mandibular movement, articular movement, and movement at occlusion of teeth.

In some variations, the mandibular movement may comprise one or more of a hinge, a protrusion, and a lateral movement. In some variations, the articular movement may comprise one or more of a rotation, a translation, a protrusive condylar path, a progressive condylar path, a laterotrusive condylar path, mediotrusive condylar path, a condylar path angle, a Bennett angle, and a Bennett movement. In some variations, the movement at occlusion of teeth may comprise one or more of an incisal path, an incisal path angle, an incisal path distance, an occlusal guidance, and an occlusal interference. In some variations, the orthodontic treatment may be planned using the jaw model of the patient.

In some variations, a plurality of aligner trays may be generated with tooth-receiving cavities, each aligner tray corresponding to a respective tooth arrangement.

In some variations, a display may be configured to display the jaw model of the patient and a user interface for navigating the jaw model.

### BRIEF DESCRIPTION OF THE DRAWINGS

FIG. 1 is a schematic illustration depicting one variation of a method for orthodontic treatment planning.

FIG. 2 is a schematic illustration depicting one variation of a system for orthodontic treatment planning.

FIGS. 3A and 3L are schematic perspective views depicting a rotation axis of a mandible of a patient. FIGS. 3B, 3D-3F, and 3K are schematic sagittal sectional views depicting the temporomandibular joint. FIG. 3C is a schematic coronal sectional view depicting the glenoid fossae. FIGS. 3G and 3H are schematic coronal sectional views depicting jaw movement. FIG. 3I is a schematic sagittal sectional view depicting jaw movement. FIG. 3J is a schematic coronal sectional view depicting the mandibular condyle.

FIG. 4A is a schematic sagittal sectional view of exemplary rotation axes of the mandible of a patient. FIG. 4B is a schematic sagittal sectional view of exemplary rotation paths of the mandible of the patient.

FIGS. 5A and 5B are schematic sagittal sectional views of an exemplary optimal rotation of a mandible of a patient.

FIGS. 6A and 6B are schematic sagittal sectional views of an exemplary sub-optimal rotation of a mandible of a patient.

FIGS. 7A and 7B are schematic sagittal sectional views of another exemplary sub-optimal rotation of a mandible of a patient.

FIG. 8 is a schematic representation of glenoid fossa morphology and mandibular translation path.

FIG. 9 is an occlusal map of centric movement of a patient.

FIG. 10 is an occlusal map of centric and excursive movements of a patient.

### DETAILED DESCRIPTION

Non-limiting examples of various aspects and variations of the invention are described herein and illustrated in the accompanying drawings.

Described herein are methods and systems for increasing the accuracy of an orthodontic treatment plan. The output of the methods and systems described herein are not only for diagnostic and treatment planning services, but may also be useful for the direct and indirect manufacturing of orthodontic appliances such as braces, functional appliances, and clear aligners. Specifically, the methods and systems described herein enable determination of a mandibular rotation axis (a rotation axis of the mandible) and glenoid fossae of the temporal bone. The rotation axis of the mandible and glenoid fossae may then be used in treatment planning on which to base orthodontic treatment according to one or more predetermined movement paths (e.g., articular, mandibular, movement at occlusion) of the jaw, thereby facilitating clinical benefits such as reducing total treatment time and reducing unnecessary root movement. For example, a jaw articulator model may be configured to model dental occlusion and the envelope of motion and occlusal function based on a volumetric morphology of the temporomandibular jaw joint (e.g., mandibular rotation axis, glenoid fossae). Additionally or alternatively, teeth movement may be modeled relative to mandible position, articulation, and the vertical, transverse, and sagittal dimensions of occlusion.

By contrast, the efficiency and effectiveness of conventional orthodontic treatment is typically dependent on the experience and skill of the practitioner. For example, conventional modeling of orthodontic and dentofacial orthopedic jaw articulation relies on manual manipulation of a facebow device to estimate jaw function. The facebow device may be configured to estimate the position of the jaw joint in relation to the teeth, the upper and lower jaws, and the position of the occlusal plane. The estimated jaw joint position may then be applied to a physical model of the teeth and mounted on a physical articulator device that is generally set in plaster. However, the estimated jaw joint position inherently introduces error as it is based on the external auditory meatus rather than the temporomandibular joint itself. Moreover, conventional modeling also relies on averages, guesses, estimations, and bite jumps that are user-defined and/or based upon libraries of averages. A bite jump models a shift of a first dental arch versus an opposing second arch for visualization of teeth and/or jaw movement. Bite jumps may occur in one, two, or three planes of space. Consequently, conventional jaw articulator models require trial-and-error adjustments that may increase the number of patient visits and extend treatment times, leading to higher costs and reduced patient satisfaction.

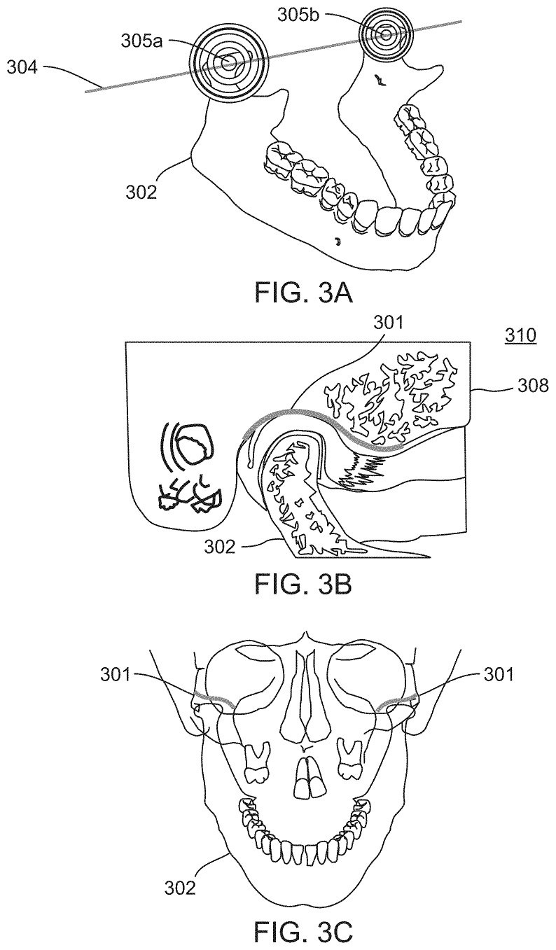

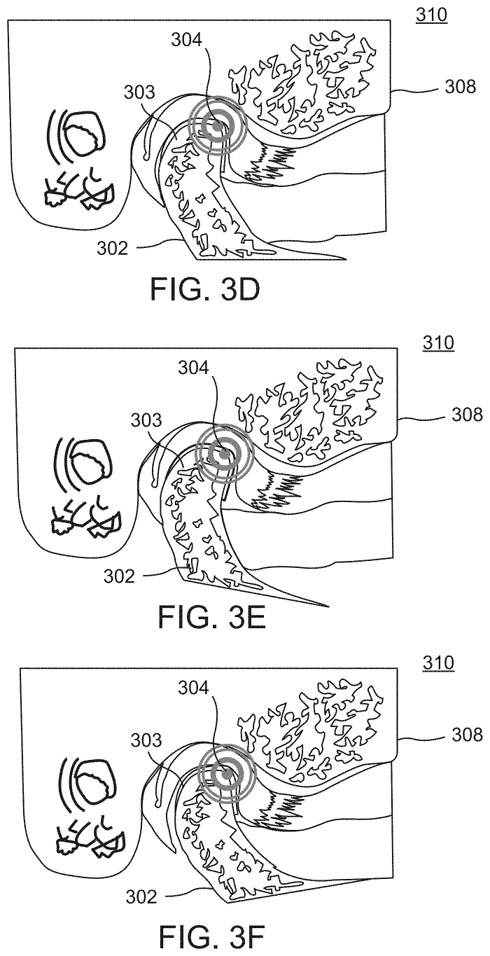

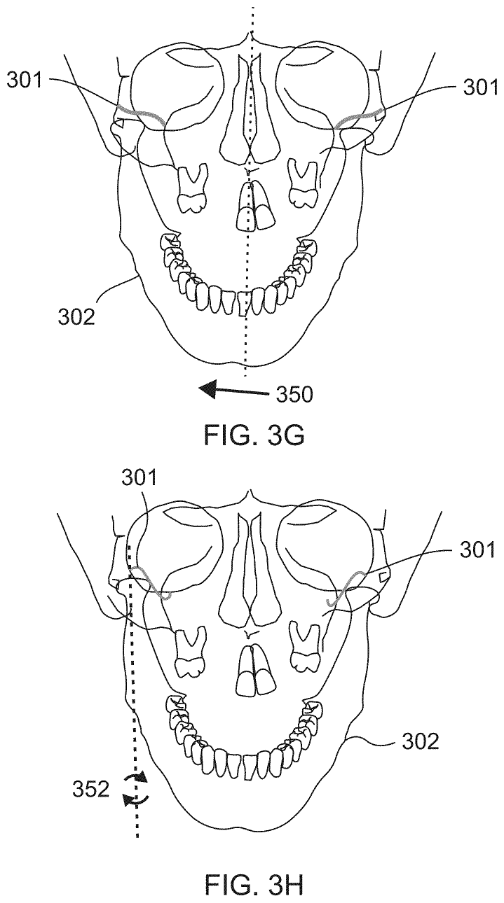

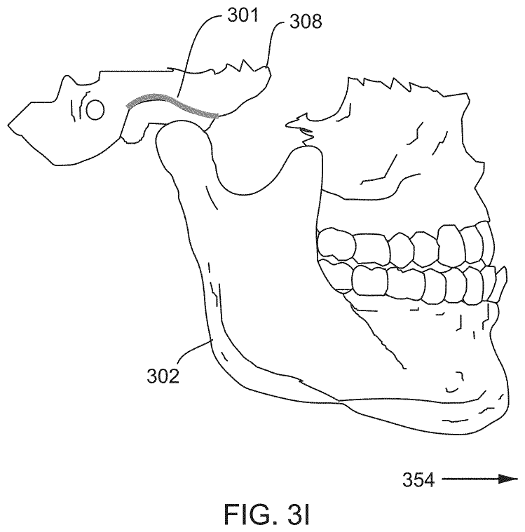

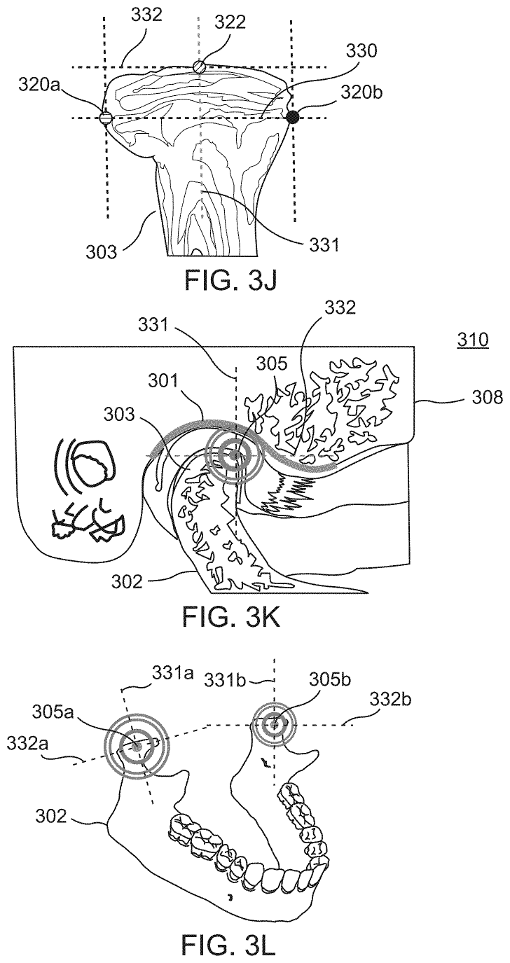

Relevant anatomy is illustrated in the schematics of FIGS. 3A-3C. FIG. 3A is a schematic perspective view depicting a mandible 302 of a patient. As further described herein, a method of orthodontic treatment planning may define a rotation axis 304 of the mandible 302 that bilaterally intersects the mandibular condyle of the mandible 302 at mandibular hinge points 305a, 305b. For example, the rotation axis 304 may intersect 305 an anterio-superior-most portion (e.g., mandibular hinge points 305a, 305b) of the mandibular condyle of the mandible 302. FIG. 3B is a schematic sagittal sectional view of the temporomandibular joint 310 comprising the glenoid fossae 301, mandible 302, and temporal bone 308. As described below in more detail with respect to FIGS. 3D-3I, the mandible 302 may move (e.g., articulate) relative to the temporal bone 308. FIG. 3C is a schematic coronal sectional view 320 depicting the mandible 302 and glenoid fossae 301.

### Methods for Orthodontic Treatment Planning

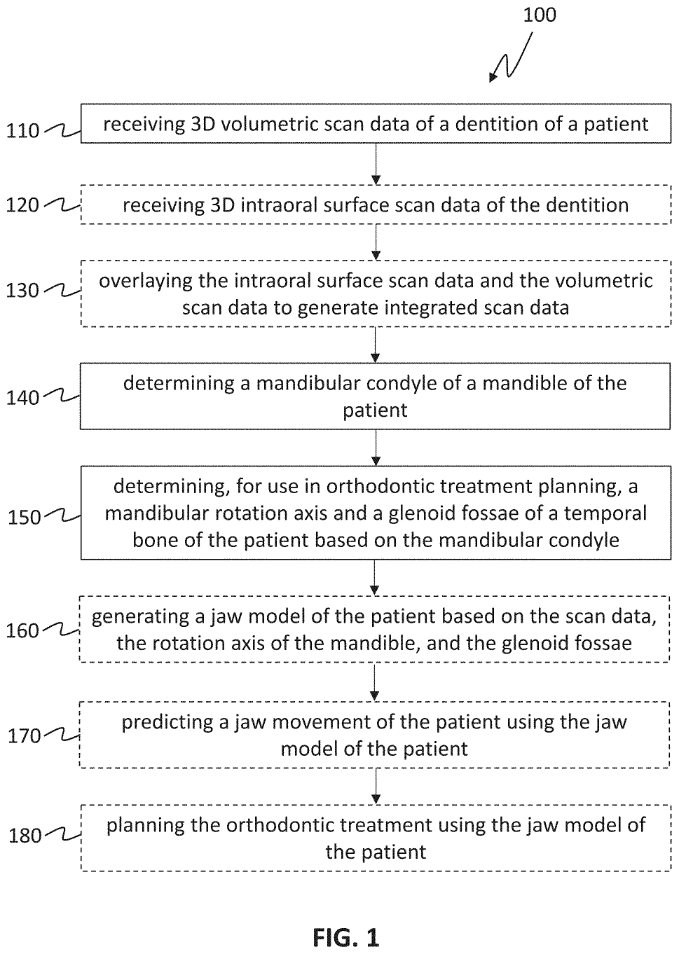

Generally, as shown in FIG. 1, in some variations, a method 100 of orthodontic treatment planning for a patient includes receiving three-dimensional volumetric scan data of a dentition of the patient 110. Optionally, three-dimensional intraoral surface scan data of the dentition of the patient 120 may be received. Optionally, the intraoral surface scan data and the volumetric scan data may be overlaid to generate integrated scan data 130. A mandibular condyle of a mandible of the patient may be determined 140. A mandibular rotation axis and a glenoid fossae of a temporal bone of the patient may be determined based on the mandibular condyle for use in planning an orthodontic treatment 150. Optionally, a jaw model of the patient may be generated based on the scan data, the rotation axis of the mandible, and the glenoid fossae 160. Optionally, a jaw movement of the patient may be predicted using the jaw model of the patient 170. Optionally, the orthodontic treatment may be planned using the jaw model of the patient 180.

### Scan Data

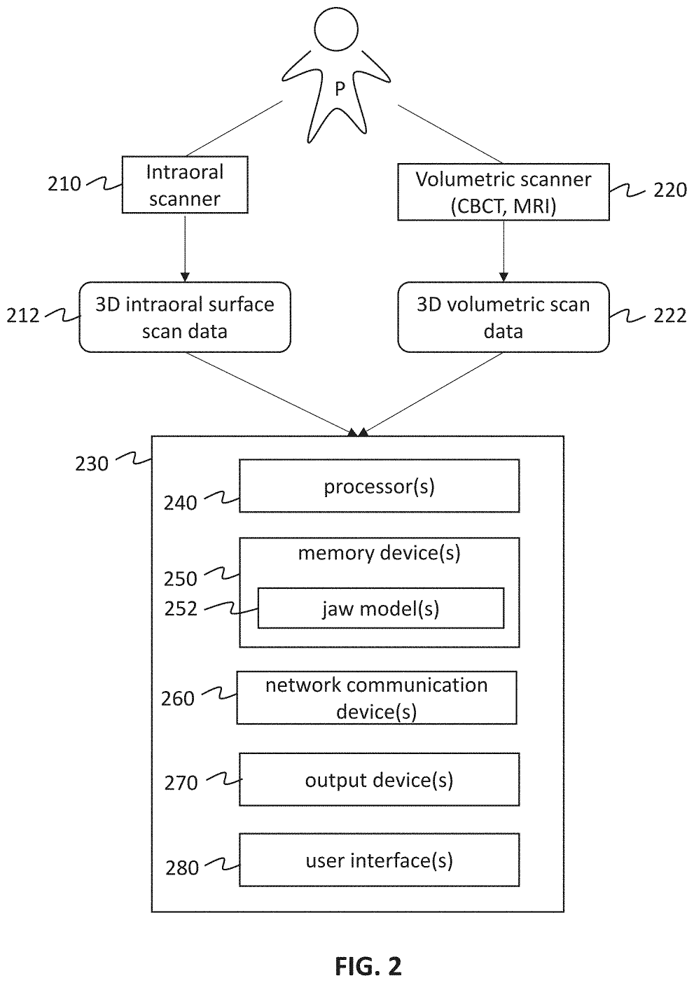

Three-dimensional volumetric scan data of a dentition of the patient may be received 110 and/or three-dimensional intraoral surface scan data of a dentition of the patient may be received 120 for processing and/or analysis. As shown in the schematic of FIG. 2, a first scan data set 212 (e.g., three-dimensional intraoral surface scan data) and a second scan data set 222 (e.g., three-dimensional volumetric scan data) may be generated by respective one or more scanning devices configured to obtain anatomical imaging data for a patient P. For example, an intraoral scanning device 210 (e.g., intraoral scanner) may be used by a practitioner or other user to obtain image data (e.g., optical color scan data) representative of external surfaces of a patient's dentition (e.g., teeth crowns, gingiva, etc.). The intraoral scanning device 210 may, for example, be a handheld scanner that emits light toward the patient's dentition as the scanner is manipulated inside the mouth of the patient. The emitted light reflects off surfaces of the patient's dentition, and the reflected light is captured by the intraoral scanner and subsequently analyzed to transform the reflected light data into surface imaging data. An exemplary intraoral scanner suitable for use in obtaining three-dimensional intraoral surface scan data 212 is the CS 3600 intraoral scanner available from CARESTREAM DENTAL LLC (Atlanta, Ga., USA). However, any suitable intraoral scanner may be used to obtain such intraoral surface scan data 212.

Generally, the digitized surfaces of the patient's dentition obtained from the intraoral surface scan may be used to create one or more patient-customized orthodontic appliances (e.g., using computer-aided design and computer-aided manufacturing (CAD/CAM) technology), which may, for example, be used to apply forces to teeth and induce controlled orthodontic tooth movement (OTM). Accurate surface scan data of a patient's teeth enable such appliances to have a predictably intimate fit to the unique curvatures of the teeth. Moreover, precise manipulation of accurate intraoral surface scan data allows the creation of orthodontic appliances to induce effective OTM.

While intraoral surface scan data may provide information about the external form of the tooth crowns and at least a portion of gingiva, the intraoral surface scan data does not supply direct information about certain other structures such as the mandible, temporal bone, and the temporomandibular joints.

In some variations, the volumetric scan data 222 may be obtained by a volumetric scanning device 220 (e.g., volumetric scanner). In some variations, the volumetric scanner 220 may provide three-dimensional X-ray imaging (e.g., cone-beam computed tomography (CBCT)) of dentition (e.g., crowns, gingiva, root structures, bone volume and density), the jaw joint, the cranium, and the viscerocranium. Specifically, the volumetric scanner may be configured to provide detailed information regarding the mandible and the temporal bone. An exemplary CBCT X-ray scanner suitable for use in obtaining three-dimensional volumetric scan data 222 is the RAYSCAN a imaging device available from RAY COMPANY (RAY AMERICA, Inc., Fort Lee, N.J., USA). However, any suitable extraoral scanners providing volumetric information of dentition and craniofacial features may be used to obtain the volumetric scan data 222. For example, the volumetric scanner 220 may comprise a magnetic resonance imaging device.

Generally, the volumetric scan data obtained from an ionizing or non-ionizing volumetric scanner may be used to identify and/or characterize patient anatomical features such as hinges, joints, bones, as well as to measure or otherwise quantify other patient characteristics. For example, information relating to the mandible may improve the modeling of orthodontic tooth movement by, for example, predicting jaw movement, as further described below. In some variations, the volumetric scan data corresponds to one or more of a cranium and viscerocranium of the patient.

### Overlaying Scan Data

In some variations, the received intraoral surface scan data and the volumetric scan data may be overlaid to generate integrated scan data 130. Generally, the scan data may be imported into a software application on a computing device for display in a user interface. Combining volumetric scan data and intraoral surface data may reduce the error introduced by artifacts present in the volumetric scan data of the teeth such as those introduced from radio-opaque dental restorative materials like amalgam, metal, composites, and the like.

Software instructions stored on a machine-readable storage medium (as described in further detail below) may enable display and manipulation of the intraoral surface scan data and the volumetric scan data on the computing device. The software instructions may, in some variations, enable registration of the intraoral surface scan data with the volumetric scan data such that both sets of scan data are aligned with each other. The registered intraoral surface scan data and volumetric scan data may share a common coordinate system, such that a resulting integrated patient model may be manipulated within the common coordinate system. Registration of the scan data may include, for example, alignment of one or more anatomical landmarks (e.g., visible crown features) and/or fiducials (e.g., radiopaque and optically visible markers in the patient's mouth and/or on the patient's dental features). Generally, the digitized surface scan and volumetric scan models may be aligned by a computational best-fit alignment algorithm, which may, for example, provide for six degrees of freedom in adjustment and scaling as needed. The best-fit algorithm may be performed separately once for the upper teeth, and once for the lower teeth.

One or both of the intraoral surface scan data and the volumetric scan data may be rescalable and/or rotatable to better facilitate the alignment and overlay of the scan data. For example, the software instructions may enable display of one or more handle icons associated with “grab points” on the scan data. Such handle icons may be manipulated (e.g., with a “click and drag” function) with a user input device such as a mouse or a touch screen, in order to rescale and/or rotate the scan data. Furthermore, the software instructions may enable selected portions of the intraoral surface scan data and/or volumetric scan data to be isolated via cropping or other similar image editing functionality.

In some variations, overlaying of the intraoral surface scan data and the volumetric scan data may be performed manually. For example, user input may manipulate one or both sets of scan data until the scan data are scaled and/or aligned appropriately. As another example, a user may select a minimum number of points per jaw in corresponding locations on the surface scan model and the volumetric scan model (e.g., three or more on each jaw, per model) as key points, and align the respective sets of key points on the models in order to overlay them into an integrated patient model. In some variations, the overlaying of the intraoral surface scan data and the volumetric scan data may be performed automatically with suitable machine vision techniques (e.g., edge detection, corner finding, etc.). In yet other variations, the overlaying of the intraoral surface scan data and the volumetric scan data may be performed semi-automatically utilizing both manual and algorithmic techniques. For example, a user may manually indicate corresponding locations on the multiple images of scan data with virtual markers (e.g., placed on distinctive malocclusions, on key points such as along the interproximal margin, along various crown outlines or gingiva boundaries, etc.), and software instructions may be executed to automatically scale the images as necessary and/or align the corresponding virtual markers to produce the overlaid set of scan data (e.g., using a suitable computational best-fit algorithm). The results of such automatic or semi-automatic operation may be further adjusted with manual input and/or require manual input to indicate approval of the automatically or semi-automatically generated integrated patient model.

After at least a portion of the intraoral surface scan data and at least a portion of the volumetric scan data are overlaid to generate integrated scan data, the integrated scan data may be displayed in a user interface on the computing device as a patient model for further use during diagnosis and/or treatment planning. For example, the patient model may be rotated for viewing in different perspectives, displayed with suitable cut-away or cross-sectional views.

In some variations, the intraoral surface scan data and the volumetric surface scan data may capture different states of the patient's dentition or may otherwise be at least partially inconsistent. For example, the intraoral surface scan and the volumetric scan may have been performed at different times, and the patient's structures (e.g., teeth, gingiva, etc.) may have moved through natural physiologic processes such as growth and remodeling, and/or by induced processes like orthodontic treatment. In these variations, such as if one or more teeth and/or jaws have moved, digital alignment of surface scan and volumetric scan models may be executed on a tooth-by-tooth basis to help ensure accurate crown-to-root alignment among the models. Furthermore, it may be helpful in some variations to segment the teeth of one of the two data sets (surface scan or volumetric scan) prior to overlay, in order to help with crown-to-root alignment. In some variations, conflicts between the intraoral surface scan data and the volumetric scan data may be resolved by setting the intraoral surface scan data as the ground truth for buccal, lingual, incisal, and occlusal dental surfaces, and for setting the volumetric scan data as the ground truth for interproximal dental surfaces and root surfaces. Suitable examples of scan data methods and systems are further described in International Publication No. WO 2020/197761, published Oct. 1, 2020, and incorporated herein by reference.

Once registration and/or alignment of the surface scan and volumetric scan models is obtained, the crown morphology supplied by any new (subsequent) surface scan may also be used to infer the new position of the roots using the actual root morphology from previous integrated scan data model(s).

### Mandibular Features Determination

Various features of the mandible and/or other anatomy of the patent may be determined from the scan data (e.g., volumetric scan data, overlaid volumetric and intraoral surface scan data, etc.). For example, in some variations, a set of mandibular condyles of a mandible of the patient may be determined using the received scan data (e.g., volumetric scan data, overlaid volumetric and intraoral surface scan data) 140. A rotation axis of the mandible and glenoid fossae of a temporal bone of the patient may be determined based on the determined mandibular condyle for use in orthodontic treatment planning 150. For example, the rotation axis of the mandible and the glenoid fossae may be used in treatment planning to predict jaw movement, and may improve the accuracy of orthodontic treatment planning that facilitates the shortening of a total treatment time.

FIG. 3D is a schematic sagittal sectional view depicting the temporomandibular joint 310 including the mandible 302, mandibular condyle 303, mandibular rotation axis 304, and temporal bone 308. FIG. 3E depicts the mandible 302 rotated about the axis 304 in an open configuration and FIG. 3F depicts the mandible 302 rotated about the axis 304 in a closed configuration. The mandible 302 may be rotated about the axis 304 in a plurality of configurations between the open and closed configuration. FIGS. 3G and 3H are schematic coronal sectional views depicting mandible 302 movement 350, 352 relative to the glenoid fossae 301. FIG. 3I is a schematic sagittal sectional view depicting mandible 302 translation relative to the glenoid fossae 301 of the temporal bone 308.

In some variations, as shown in the schematic perspective view of FIG. 3A, the mandibular rotation axis 304 may be defined by mandibular hinge points 305a, 305b connecting the two anterior-superior-most poles of the condylar heads of the mandible 302 interfacing against a temporomandibular joint disk (not shown). In some variations, the mandibular rotation axis 304 and the glenoid fossae 301 may be based on the anterio-superior-most portion of the mandibular condyles 303 (e.g., FIGS. 3D-3F, 3K) specified bilaterally and determined as described in more detail below.

In some variations, each mandibular condyle 303 of the mandible 302 may be determined using the received scan data. For example, each mandibular condyle 303 may be determined using one or more of model segmentation (e.g., of scan data input to a machine learning model) and/or manual input. In some variations, discrete portions of a model corresponding to the surface scan data and/or a model corresponding to the volumetric scan data may be identified in a model segmentation process. For example, different portions of a model, where the different portions correspond to different anatomical features, may be segmented. For example, the mandible in the model may be segmented in order to enable independent selection, viewing, and/or manipulation of each portion of the mandible in isolation. In some variations, at least each mandibular condyle of the mandible may be separated (as a discrete, identifiable volume) from the rest of the model. Additionally or alternatively, the model may be segmented to separate other anatomical features such as the coronoid process, body, teeth, temporal bone, and the like.

Furthermore, in some variations, model segmentation may be automatically performed based at least in part on voxel density of various voxels in the volumetric scan model. Different kinds of patient tissue will be represented with different voxel density in the volumetric scan data, as the result of the differing radiopacity of different kinds of tissue. For example, bones have relatively higher radiopacity than gingiva, and therefore will be represented with greater voxel density than gingiva in a CBCT scan. As another example, tooth enamel and root dentin have a higher radiopacity than their surrounding alveolar bone, and will be represented with greater voxel density than surrounding bone in the volumetric scan data. Accordingly, in some variations, different regions of a model may be automatically identifiable by monitoring threshold changes in voxel density across neighboring voxels in the integrated patient model, thereby aiding segmentation.

In some variations, partial or full segmentation of both the surface scan model and the volumetric scan model may be performed prior to overlaying the models to form a model. In some variations, partial or full segmentation of the integrated patient model may be performed after overlaying (at least partially) unsegmented surface scan and volumetric scan models. In yet other variations, either the surface scan model or the volume scan model may be segmented after their overlay, based at least in part on alignment information derived from the model.

FIG. 3J is a schematic coronal sectional view depicting a mandibular condyle 303. A lateral-most point (e.g., 320a) and a medial-most point (e.g., 320b) of the condyle 303 may be determined (e.g., using machine vision techniques) and used to define a first axis 330 therethrough that connects the lateral-most and medial-most points of the condyle 303. A superior-most point 322 of the condyle 303 may be determined from the scan data, such as by analyzing a sequence of plane slices perpendicular to the axis 330 (e.g., planes taken in series in a medial-lateral direction) and identifying the point 322 on such planes where the surface of the condyle is located most superior relative to the rest of the condyle surface. At this superior-most point 322, a second axis 331 perpendicular to the first axis 330 and which intersects the superior-most point 322 may be defined. Furthermore, a plane 332 may be defined which is parallel to the first axis 330 and perpendicular to the second axis 331, where the plane 332 includes the superior-most point 322 of the condyle.

As shown in the schematic sagittal sectional view of FIG. 3K, a mandibular hinge point 305 may be determined as the anterior-most point of the condyle 303 along the plane 332, where the mandibular hinge point 305 is the anterio-superior-most point of the condyle as described herein. Furthermore, as shown in FIG. 3L, mandibular hinge points 305a, 305b may be separately determined for each side of the mandible 302 using respective second axes 331a, 331b, and planes 332a, 332b. As shown in FIG. 3A, the mandibular rotation axis 304 intersects each of the mandibular hinge points 305a, 305b. The methods described herein for determining the mandibular rotation axis 304 are advantageous over conventional techniques. For example, conventional techniques fail to accurately and precisely model patient-specific anatomy, at least because each of the left and right condyles of a particular patient may have different medial and lateral pole orientations and morphology such that correct determination of the mandibular hinge points may be difficult using 2D X-rays due to artifacts (e.g., due to superimposition). Likewise, conventional facebow transfers estimate the jaw joint based on external morphology which may not correspond to the patient's jaw joint anatomy due to developmental differences and remodeling due to disease and function. In contrast, by utilizing the methods and systems herein to define the mandibular rotation axis 304, more accurate and precise representation of anatomy and modeling of jaw movement for a patient may be achieved.

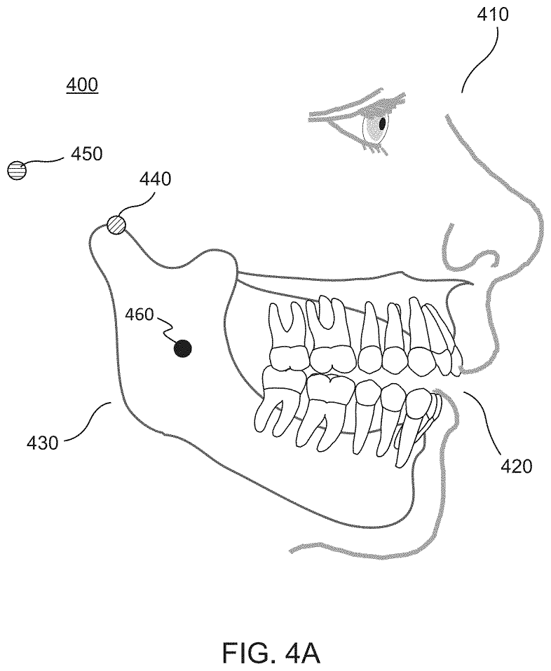

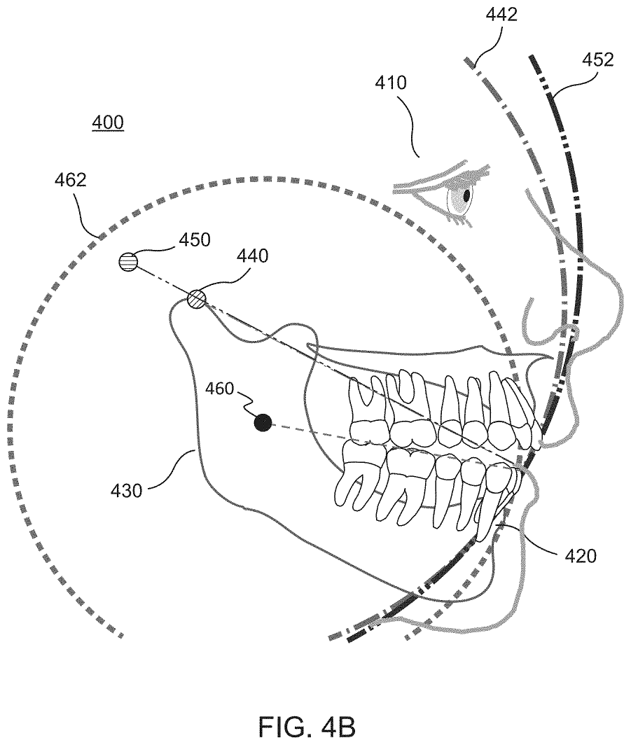

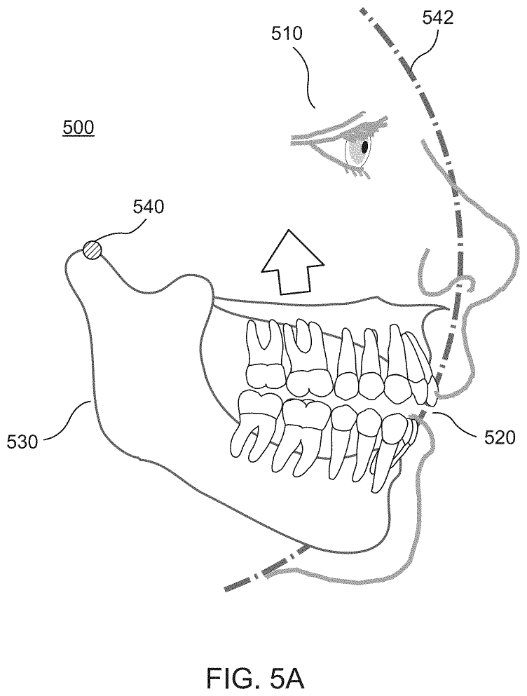

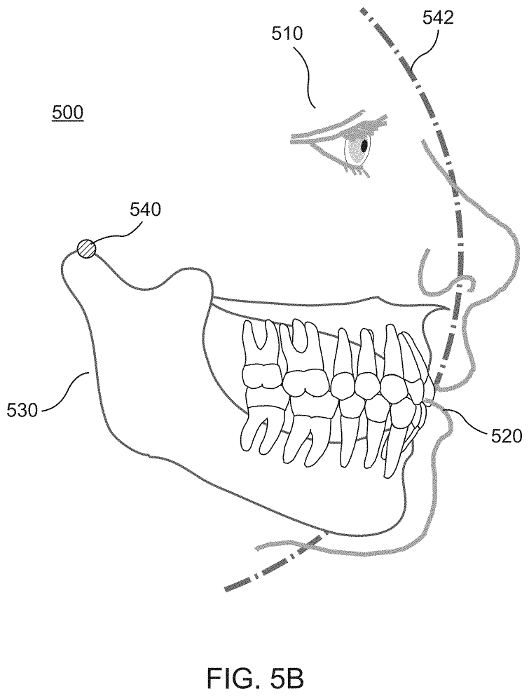

In some variations, a shape of the glenoid fossae may be determined using one or more machine vision techniques. As shown in FIGS. 3D-3F, an anterio-superior-most portion of the mandibular condyle 303 corresponds to a rotation axis 304 of the mandible 302. Similarly, rotation axis 440 of mandible 430 in FIGS. 4A and 4B and rotation axis 540 of mandible 530 in FIGS. 5A and 5B correspond to a rotation axis of the mandible at an anterio-superior-most portion of the mandibular condyle.

In some variations, one or more of the mandibular condyle, mandibular rotation axis, and the glenoid fossae may be defined manually based on user input (e.g., by placement of one or more markers on the volumetric scan data), or otherwise indicate. For example, a user may visually inspect the mandible in at least one sagittal view of the volumetric scan data, and mark or adjust a mandibular rotation axis that intersects the anterio-superior-most portion of the mandibular condyle.

In some variations, one or more of the mandibular condyle and glenoid fossae may be automatically determined based on software-instructed analysis of the scan data. For example, different anatomical regions (e.g., mandible, mandibular condyle, temporal bone, glenoid fossae, crown, root, periodontal ligament, bone, etc.) may be identified using a machine learning model as described in more detail herein. For example, the scan data may be segmented into different anatomical regions for display and/or analysis. In view of such identification of these anatomical regions, total volume and overall shape of the jaw may be automatically determined using the scan data input to a machine learning model (e.g., jaw model 252 stored in memory device 250).

Any one of the above-described variations of determining one or more of the mandibular condyle and glenoid fossae may be automatically executed, or presented to a user (e.g., within a software application) as options for selection. Furthermore, in some variations, two or more of the above-described variations of determining one or more of the mandibular condyle and glenoid fossae may be performed, and their results may be averaged. Additionally or alternatively, any one or more of the above-described variations of determining one or more of the mandibular condyle and glenoid fossae may be performed, and the resulting location may be manually adjusted.

### Machine Learning

Systems, devices, and methods described herein may implement machine learning models to process and/or analyze image data regarding a patient's anatomy. Such machine learning models may be configured to identify and differentiate (e.g., segment) between different anatomical parts within anatomical structures. In some variations, machine learning models described herein may include, but are not limited to, neural networks, including deep neural networks with multiple layers between input and output layers. For example, one or more convolutional neural networks (CNNs) may be used to process patient image data and produce outputs classifying different objects within the image data (e.g., 3D volumetric scan data, 3D intraoral surface scan data). While certain examples described herein employ CNNs, it can be appreciated that other types of machine learning algorithms can be used to process scan data, including, for example, support vector machines (SVMs), decision trees, k-nearest neighbor, and artificial neural networks (ANNs).

In some variations, an anatomical parts models may include one or more machine learning models configured to identify (e.g., segment) a set of anatomical parts (e.g., mandibular condyle, glenoid fossae). In some variations, anatomical parts data may include information relating to anatomical parts of a patient for identifying, characterizing, and/or quantifying different features of one or more anatomical parts, such as, for example, a location, color, shape, geometry, or other aspect of an anatomical part. The anatomical parts data may enable a processor to perform anatomical parts identification based on scan data inputted to the anatomical parts model.

Systems, devices, and methods described herein may use a neural network and deep learning based approach to identify (e.g., segment) anatomical parts of interest using a training dataset including images with labeled anatomical parts. In some variations, one or more machine learning models may be trained using training datasets including input scan data and labels representing desired outputs. The machine learning models may use the training datasets to learn relationships between different features in the scan data and the output labels. One or more methods of training a model may be used including supervised learning, unsupervised learning, semi-supervised learning, and reinforcement learning. For example, a supervised learning model may include a neural network model, feedforward neural network (FNN), a recurrent neural network (RNN), a convolutional neural network (CNN), a deep learning model, a support vector machine, a naive Bayes model, a decision tree, or a k-nearest neighbor algorithm.

In some variations, the training dataset may include input scan data of anatomical structures and corresponding output scan data of anatomical structures with labelling applied to different parts of the anatomical structures. The images of the scan data may be grouped into multiple batches for training the anatomical parts model. For example, each image within a batch may include images representative of a series of slices of a 3D volume of an anatomical structure. Each output image may include at least one label which identifies a portion of that image as corresponding to a specific anatomical part. In some variations, each output image may include a plurality of labels, with the plurality of labels indicating different parts of the patient anatomy.

Data augmentation may be performed on the training dataset to create a more diverse set of images. Each input image and its corresponding output image may be subjected to the same data augmentation, and the resulting input and output images may be stored as new images within the training dataset. The data augmentation may include applying one or more transformations or other data processing techniques to the images. These transformations or processing techniques may include: rotation, scaling, movement, horizontal flip, additive noise of Gaussian and/or Poisson distribution and Gaussian blur, etc.

In some variations, an anatomical parts model may be trained using the training dataset, including the original scan data and/or the augmented scan data. In some variations, the training may be supervised. For example, the training may include inputting the input images into the anatomical parts model, and minimizing differences between an output of the anatomical parts model and the output images (including labeling) corresponding to the input images. In some variations, the anatomical parts model may be a CNN model, whereby one or more weights of a function may be adjusted to better approximate a relationship between the input images and the output images.

In some variations, a validation dataset may be used to assess one or more performance metrics of the trained anatomical parts model. Similar to the training dataset, the validation dataset may include input images of anatomical structures and output images including labelled anatomical parts within the anatomical structures. The validation dataset may be used to check whether the trained anatomical parts model has met certain performance metrics or whether further training of the anatomical parts model may be necessary.

In some variations, systems, devices, and methods described herein may perform pre-processing of patient integrated scan data prior to performing anatomical part identification to remove noise prior to performing anatomical part identification. Alternatively or additionally, the one or more images may be processed using other techniques, such as, for example, filtering, smoothing, cropping, normalizing, resizing, etc.

In some variations, the scan data (e.g., integrated scan data) may be input into an anatomical parts model. In instances where the anatomical parts model is implemented as a CNN, the input scan data may be passed through the layers of the CNN. The anatomical parts model may return outputs on the scan data. Optionally, the output of the anatomical parts model may be postprocessed, e.g., using linear filtering (e.g., Gaussian filtering), non-linear filtering, median filtering, or morphological opening or closing.

In some variations, the output of the anatomical parts model may include per-class probabilities for each pixel (or group of pixels) of each image of the image data. For example, the anatomical parts model may be configured to classify the image data into one of a plurality of classes. Accordingly, the anatomical parts model may be configured to generate, for each pixel or group of pixels in the images, the probability that a pixel or group of pixels belongs to any one of the classes from the plurality of classes. The plurality of classes may correspond to a plurality of anatomical parts (e.g., mandibular condyle, glenoid fossae).

### Jaw Model

In some variations, a jaw model of the patient may be generated based on the scan data, the rotation axis of the mandible, and the glenoid fossae of the temporal bone 160. FIG. 4A is a schematic sagittal sectional view of a jaw model 400 comprising exemplary rotation axes 440, 450, 460 of a mandible 430 of a patient 410. FIG. 4B is a schematic sagittal sectional view of exemplary rotation paths 442, 452, 462 corresponding to respective rotation axes 440, 450, 460 of the mandible 430 of the patient 410. For example, the rotation paths 442, 452, 462 depict the path of the anterior mandibular dental archform with respect to a vertical dimension of occlusion.

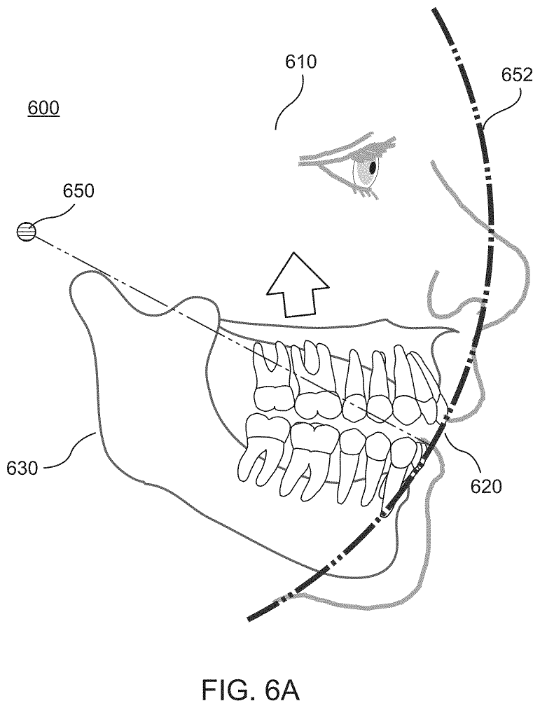

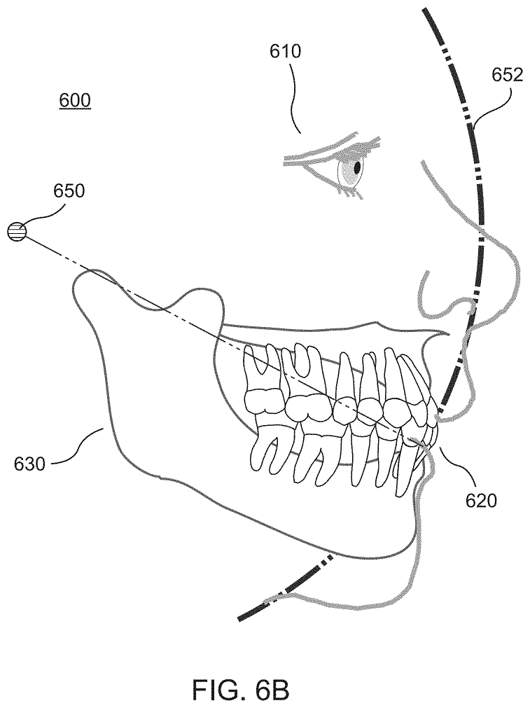

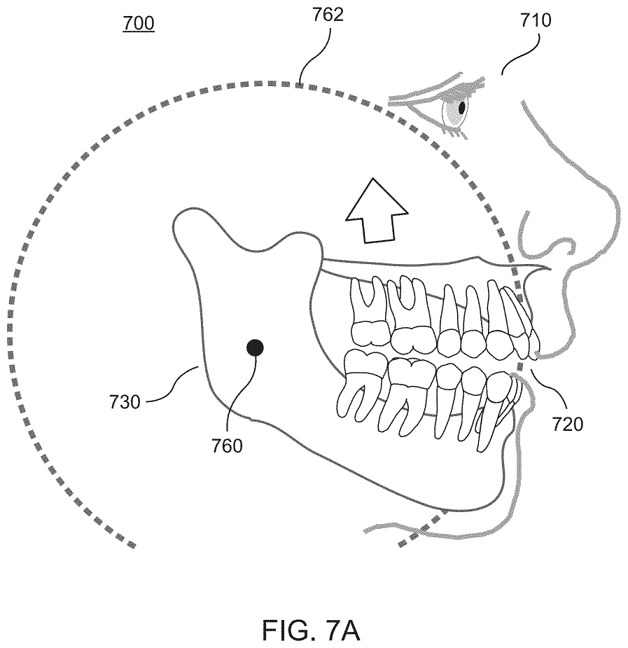

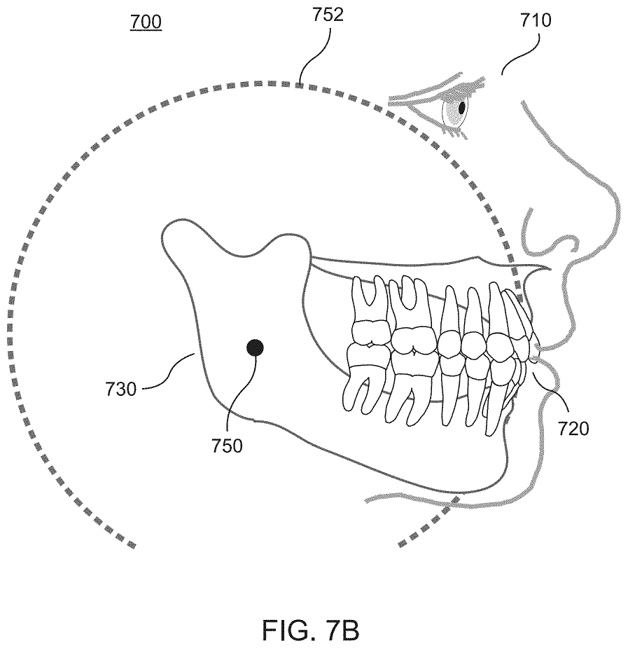

First rotation axis 440 is a more correct and appropriate mandibular rotation axis compared to the rotation axes 450 and 460. Specifically, the first rotation axis 440 corresponds to a mandibular condyle of the mandible (e.g., rotation axis 440 is located at an anterio-superior-most portion of the mandibular condyle), as determined and described in detail herein with respect to method 100. Second rotation axis 450 corresponds to an external auditory meatus, as typically determined based on, for example, conventional physical modeling techniques such as with a facebow device coupled externally to the patient's head. Third rotation axis 460 corresponds to an incorrect determination of the mandibular rotation axis that is shorter and inferior to the first rotation axis 440. Predicted jaw movements and corresponding teeth 420 positioning may vary significantly if the determined rotation axis deviates from the true rotation axis 440 toward either the second rotation axis 450 or third rotation axis 460, as described in more detail with respect to FIGS. 6A-7B.

FIGS. 5A and 5B are schematic sagittal sectional views of a jaw model 500 comprising an optimal rotation axis 540 of a mandible 530 of a patient 510 through an anterio-superior-most portion of the mandibular condyles (e.g., similar to rotation axis 440 shown in FIGS. 4A and 4B). As the teeth 520 are brought together along the path 542 from FIG. 5A to 5B, the model 500 depicts an accurate prediction of articulation and bite correction.

FIGS. 6A and 6B are schematic sagittal sectional views of a jaw model 600 having a conventional sub-optimal (e.g., inaccurate) rotation axis 650 of a mandible 630 of a patient 610 (e.g., similar to rotation axis 450 shown in FIGS. 4A and 4B). For example, the mandibular rotation axis 650 corresponds to a location of an external auditory meatus that may result in dental and orthodontic appliances that poorly predict occlusal contact and bite changes due to inaccurate changes in tooth position. In particular, the center of the external auditory meatus may not correspond to a location of the mandibular rotation axis because of natural variations in patients due to developmental differences and disease, and thus functions as a poor proxy for a mandibular rotation axis. For example, the external auditory meatus is commonly located at a distance greater than about 10 mm posteriorly from a true mandibular rotation axis (e.g., rotation axis 440, 540, 640). Based on the mandibular rotation axis 650, dental movements that open the bite will swing the jaw along an arc with a larger radius with a center that is anterior to the true mandibular rotation axis (e.g., rotation axis 440, 540, 640). Furthermore, the path of the anterior mandibular dental archform in FIG. 6A increases in the vertical dimension of occlusion relative to the true mandibular rotation axis 540. With respect to orthodontic treatment planning, the sub-optimal rotation path 652 (e.g., FIG. 6B) may result in an overestimation of the forward position of the mandible 630 that may lead to unnecessarily complex or invasive procedures (e.g., such as tooth extraction) based on the incorrectly predicted tooth movement.

FIGS. 7A and 7B are schematic sagittal sectional views of a jaw model 700 having a sub-optimal (e.g., inaccurate) rotation axis 760 of a mandible 730 of a patient 710 (e.g., similar to rotation axis 460 shown in FIGS. 4A and 4B). Based on the mandibular rotation axis 760, dental movements that open the bite will swing the jaw along an arc with a smaller radius with a center that is inferior to the true mandibular rotation axis (e.g., rotation axis 440, 540). Furthermore, the path of the anterior mandibular dental archform decreases in the vertical dimension of occlusion relative to the true mandibular rotation axis 540. With respect to orthodontic treatment planning, the sub-optimal rotation path 762 may result in an underestimation of the forward position of the mandible 730 that may lead to an inaccurate bite prediction. Compared to the ideal overjet and Angle Class I occlusion as accurately modeled in FIG. 5B, the sub-optimal rotation axis 760 predicts an articulated occlusion having increased incisor over jet and an Angle class II cusp-to-cusp sagittal occlusion.

In some variations, the computing device 230 may comprise a display 270 such as a display configured to display the jaw model of the patient and a user interface for navigating the jaw model. For example, the jaw model may be displayed in a user interface on the computing device for further use during diagnosis and/or treatment planning. For example, the jaw model may be rotated for viewing in different perspectives, displayed with suitable cut-away or cross-sectional views. The user may input one or more jaw movement parameters to predict (e.g., simulate) jaw movement(s) such as shown in, for example, FIGS. 3E-3I, 5A, 5B, 9 and 10, and as described in more detail herein.

### Jaw Movement Prediction

In some variations, a jaw movement of the patient may be predicted using the jaw model of the patient 170. For example, the jaw model may be used to predict movement of one or more of the mandible, jaw joint, and occlusion based on the patient's individual physiology, morphology, and prescribed tooth movement. This may be helpful in planning an orthodontic treatment, as described in more detail herein. For example, FIGS. 3E and 3F depict the mandible 302 rotated to respective open and closed configurations about the mandibular rotation axis 304. FIG. 3G depicts articular movement 350 of the jaw, and FIG. 3H depicts a combination of movement 352 including rotation, shifting, lateral translation, and orbiting of the mandible 302. FIG. 3I depicts articular translation 354 of the mandible 302. In some variations, one or more jaw movement predictions may be displayed for a user and/or navigated using a user interface such as a graphical user interface. For example, a practitioner (e.g., user) may select one or more views and/or movements for prediction based on the jaw model of the patient.

In some variations, predicting the jaw movement may comprise one or more of a mandibular movement, articular movement, and movement at occlusion of teeth. In some variations, the jaw movement may be characterized using one or more jaw movement parameters. For example, the mandibular movement may comprise one or more of a hinge, a protrusion, and a lateral movement (e.g., Bennett's mandibular slide shift). In some variations, the articular movement may comprise one or more of a rotation, a translation, a protrusive condylar path, a progressive condylar path, a laterotrusive condylar path, mediotrusive condylar path, a condylar path angle, a Bennett angle, and a Bennett movement. In some variations, the movement at occlusion of teeth may comprise one or more of an incisal path, an incisal path angle, an incisal path distance, an occlusal guidance, and an occlusal interference.

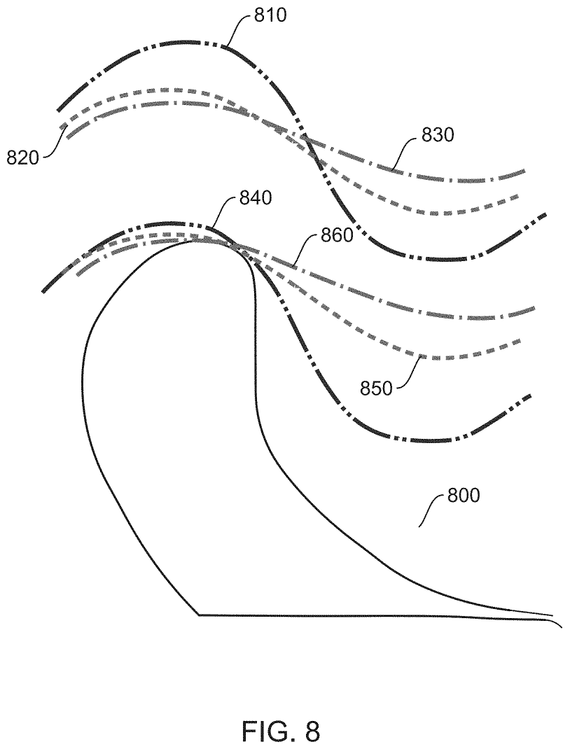

Generally, jaw movement based on a ginglymoarthrodial joint may comprise rotation and/or translation that is dependent on a physical morphology of the glenoid fossa. Moreover, movement (other than rotation) of the mandibular condyles along a mandibular rotation axis may be based on one or more of the morphology of the temporomandibular joint, the entoglenoid process of the glenoid fossa, and the articular eminence of the glenoid fossa. For example, FIG. 8 is a schematic representation of articular eminence morphology 810, 820, 830 and a condylar pole movement path 840, 850, 860 with respect to a mandibular condyle 800. The articular eminence comprises a portion of the temporal bone on which the condylar process slides during mandibular movements. FIG. 8 depicts an exemplary first articular eminence shape 810 (e.g., having a relatively steep slope), a second articular eminence shape 820 (e.g., having a relatively moderate slope), and a third articular eminence shape 830 (e.g., having a relatively flat slope) having a respective first condylar pole movement path 840 (e.g., having a relatively steep slope), a second condylar pole movement path 850 (e.g., having a relatively moderate slope), and a third condylar pole movement path 860 (e.g., having a relatively flat slope). Protrusion and laterotrusion has increased posterior disarticulation for a steeper articular eminence, which may alter registered occlusal interferences and contacts.

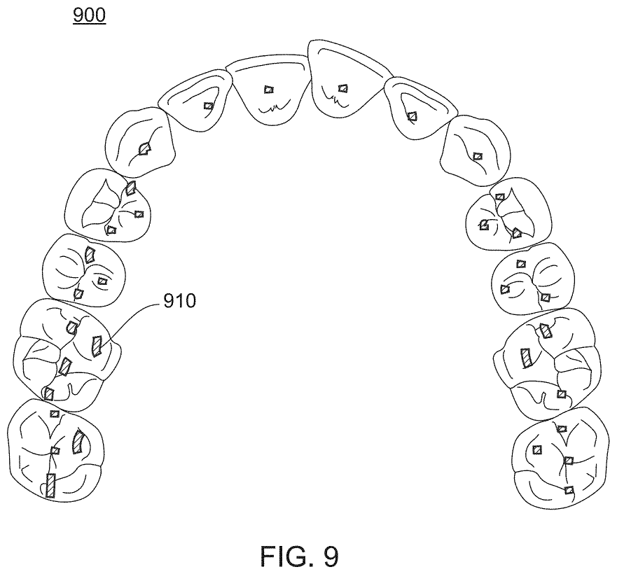

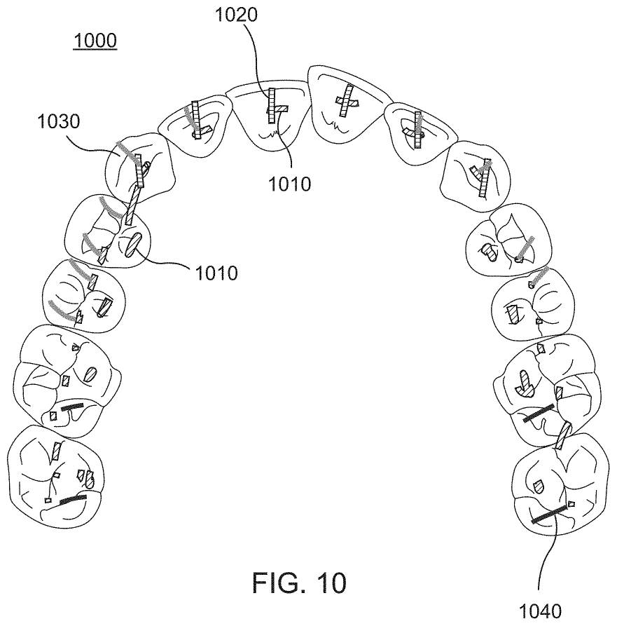

In some variations, predicted occlusal contact and/or a functional path of the teeth based on the jaw model of the patient may be visualized using an occlusal map of centric and/or excursive movements. An occlusal map may predict one or more movements comprising lateral, protrusion, retrusion, vertical, and combinations thereof by marking occlusal interferences upon the working surfaces of the teeth. For example, FIG. 9 depicts a “snap-to-fit” closing of the jaw along the mandibular rotation axis. Specifically, FIG. 9 is an occlusal map 900 of centric movements of a patient including centric occlusion/relation 910 of centric contacts via the mandibular rotation axis (not shown). FIG. 10 is an occlusal map 1000 of centric and excursive movements of a patient including centric occlusion/relation 1010 and excursive movement occlusal contacts 1020, 1030, 1040. Excursive movements comprise protrusion 1020, laterotrusion 1030, and non-working side/interferences 1040.

### Treatment Planning

In some variations, orthodontic treatment may be planned using the jaw model of the patient 180. As described herein, utilizing the jaw model of the patient enables greater accuracy in identifying the true mandibular rotation axis and glenoid fossae of a patient, thereby improving accuracy of orthodontic treatment planning and reducing overall orthodontic treatment times compared to conventional treatment planning methods. Accordingly, in some variations the methods described herein may include generating a suitable treatment plan based at least in part on the determined mandibular rotation axis and glenoid fossae of the temporal bone.

For example, in some variations, the jaw model of the patient may incorporate the determined mandibular rotation axis and/or glenoid fossae, and function to virtually model and articulate various jaw movements such as that described above, for each of various contemplated orthodontic treatment stages. For example, jaw movements may be simulated for patient dentition at each contemplated treatment stage, allowing for iterations of suitable tooth arrangements at each treatment stage as appropriate to optimize orthodontic treatment. As such, use of such a virtual jaw model may help produce a more accurate, customized treatment plant, which in turn may lead to a more personalized care that is more comfortable, efficient, and/or shorter duration. Additionally, the virtual jaw model may reduce overall guesswork and reduce the number of in-person doctor visits required for adjustments, etc. Furthermore, by incorporating root data and other information available from the volumetric scan data, use of the virtual jaw model may also help reduce side effects, risks, and/or unnecessary root movement since movement can be constrained to that which is most biologically sensible. Examples of taking into account tooth root information are described in detail in International Publication No. WO 2020/197761, which was incorporated by reference above.

The virtual jaw model may be used in planning any suitable kind of orthodontic treatment (e.g., wires and brackets, aligner trays, etc.). In some variations, treatment planning may include generating a series of one or more aligner trays with tooth-receiving cavities, each aligner tray corresponding to a respective tooth arrangement such that a patient wearing the series of aligner trays in a particular sequential order (e.g., one tray per one week, two weeks, three weeks, or other suitable period of time) experiences a gradual transition of their dentition from an original tooth arrangement to a desired or targeted tooth arrangement. The forms of the aligner trays may correspond to different stages that gradually move each of one or more teeth around a respective center of rotation determined as described above. For example, each aligner tray may to a respective tooth arrangement such that the series of aligner trays progressively move teeth in treatment paths in accordance with their true jaw movement that may close/open the vertical dimension of occlusion, minimize excursive interferences, and/or increase the number of teeth in occlusion. The aligner trays may, for example, be formed from rigid or semi-rigid polymer (e.g., through vacuum forming, injection molding, 3D printing, etc.). The aligner trays may be provided to a patient individually (e.g., shipped one at a time according to predetermined intervals) or in one or more sets.

### Systems for Orthodontic Treatment Planning

FIG. 2 illustrates various components of an exemplary system for orthodontic treatment planning. Specifically, an exemplary system may include a general computing device 230 including one or more processors 240, one or more memory devices 250, one or more network communication devices 260, one or more output devices 270, and/or one or more user interfaces 280. Exemplary general computing devices include a desktop computer, laptop computer, and mobile computing devices (e.g., tablets, mobile phones).

The processor 240 may be any suitable processing device configured to run and/or execute a set of instructions or code, and may include one or more data processors, image processors, graphics processing units, physics processing units, digital signal processors, and/or central processing units. The processor may be, for example, a general purpose processor, a Field Programmable Gate Array (FPGA), an Application Specific Integrated Circuit (ASIC), and/or the like. The processor may be configured to run and/or execute application processes and/or other modules, processes and/or functions associated with the system and/or a network associated therewith. The underlying device technologies may be provided in a variety of component types (e.g., MOSFET technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like.

In some variations, the memory 250 may include a database and may be, for example, a random access memory (RAM), a memory buffer, a hard drive, an erasable programmable read-only memory (EPROM), an electrically erasable read-only memory (EEPROM), a read-only memory (ROM), Flash memory, and the like. The memory may store instructions to cause the processor to execute modules, processes, and/or functions such as scan data processing and alignment. In some variations, the memory 250 may receive intraoral surface scan data 212 and/or volumetric scan data 222 in full (e.g., DICOM files generated by scanner-specific software). Additionally or alternatively, the memory 250 may receive intraoral surface scan data 212 and/or volumetric scan data 222 in parts, such as in a real-time or near real-time feed of data directly from the intraoral scanner 210 and/or volumetric scanner 220. In some variations, the memory 25 may store one or more machine learning models such as a jaw model 252 configured to segment patient anatomy using one or more of the volumetric scan data 222 and intraoral surface scan data as input.

Some variations described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes.

Examples of non-transitory computer-readable media include, but are not limited to, magnetic storage media such as hard disks, floppy disks, and magnetic tape; optical storage media such as Compact Disc/Digital Video Discs (CD/DVDs); Compact Disc-Read Only Memories (CDROMs), and holographic devices; magneto-optical storage media such as optical disks; solid state storage devices such as a solid state drive (SSD) and a solid state hybrid drive (SSHD); carrier wave signal processing modules; and hardware devices that are specially configured to store and execute program code, such as Application-Specific Integrated Circuits (ASICs), Programmable Logic Devices (PLDs), Read-Only Memory (ROM), and Random-Access Memory (RAM) devices. Other variations described herein relate to a computer program product, which may include, for example, the instructions and/or computer code disclosed herein.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

Furthermore, one or more network communication devices 260 may be configured to connect the general computing device to another system (e.g., intraoral scanner 210, volumetric scanner 220, Internet, remote server, database, etc.) by wired or wireless connection. In some variations, the general computing device may be in communication with one or more other general computing devices via one or more wired or wireless networks. In some variations, the communication device may include a radiofrequency receiver, transmitter, and/or optical (e.g., infrared) receiver and transmitter configured to communicate with one or more device and/or networks. In an exemplary variation, the network communication devices 260 may include a cellular modem (e.g., 3G/4G cellular modem) such that it is advantageously not dependent on internet Wireless Fidelity (WiFi) access for connectivity.

Alternatively, wireless communication may use any of a plurality of communication standards, protocols, and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, WiFi, voice over Internet Protocol (VoIP), or any other suitable communication protocol. In some variations, the devices herein may directly communicate with each other without transmitting data through a network (e.g., through NFC, Bluetooth, WiFi, RFID, and the like). For example, devices (e.g., one or more computing devices, an intraoral scanner 210, and/or a volumetric scanner 220, etc.) may directly communicate with each other in pairwise connection (1:1 relationship), or in a hub-spoke or broadcasting connection (“one to many” or 1:m relationship). As another example, the devices (e.g., one or more computing devices and/or intraoral scanner 210, and/or volumetric scanner 220, etc.) may communicate with each other through mesh networking connections (e.g., “many to many”, or m:m relationships), such as through Bluetooth mesh networking.

As described above, the computing device in the system may include one or more output devices 270 such a display and/or audio device for interfacing with a user. For example, an output device may include a display that permits a user to view the integrated patient model, treatment planning steps, and/or other suitable information related to diagnosis and/or treatment planning for orthodontic treatment. In some variations, an output device may comprise a display device including at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, and/or holographic display. In some variations, an audio device may comprise at least one of a speaker, piezoelectric audio device, magnetostrictive speaker, and/or digital speaker.

The computing device may further include one or more user interfaces 280. In some variations, the user interface may comprise an input device (e.g., touch screen) and output device (e.g., display device) and be configured to receive input data. Input data may include, for example, a selection of image scan data (e.g., for rotation, cross-sectional viewing, segmenting and/or other suitable manipulation), a selection or placement of markers (e.g., to facilitate registration of surface scan data and volumetric scan data and/or facilitate model identification as described above) and/or other interaction with a user interface. For example, user control of an input device (e.g., keyboard, buttons, touch screen) may be received by the user interface and may then be processed by the processor and memory. Some variations of an input device may comprise at least one switch configured to generate a control signal. For example, an input device may comprise a touch surface for a user to provide input (e.g., finger contact to the touch surface) corresponding to a control signal. An input device comprising a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In variations of an input device comprising at least one switch, a switch may comprise, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive user movement data from an optical sensor and classify a user gesture as a control signal. A microphone may receive audio data and recognize a user voice as a control signal.

### Exemplary Embodiments

Embodiment A1. A method of orthodontic treatment planning for a patient, the method comprising:
receiving three-dimensional volumetric scan data of a dentition of the patient; and
determining, for use in planning an orthodontic treatment, a mandibular rotation axis and a glenoid fossae of the temporal bone based on a mandibular condyle of the patient using the scan data.

Embodiment A2. The method as in any preceding Embodiment, further comprising:
receiving three-dimensional intraoral surface scan data of the dentition; and
overlaying the intraoral surface scan data and the volumetric scan data to generate integrated scan data.

Embodiment A3. The method as in any preceding Embodiment, wherein overlaying the intraoral surface scan data and the volumetric scan data comprises registering the intraoral surface scan data with the volumetric scan data.

Embodiment A4. The method as in any preceding Embodiment, wherein the volumetric scan data comprises one or more of X-ray scan data and magnetic resonance imaging scan data.

Embodiment A5. The method as in any preceding Embodiment, wherein the volumetric scan data corresponds to a cranium and viscerocranium of the patient.

Embodiment A6. The method as in any preceding Embodiment, wherein the intraoral surface scan data comprises optical color scan data.

Embodiment A7. The method as in any preceding Embodiment, wherein the mandibular rotation axis and the glenoid fossae are based on a lateral, medial, superior, and anterior geometry of the mandibular condyle.

Embodiment A8. The method as in any preceding Embodiment, wherein the mandibular rotation axis and the glenoid fossae are based on the anterio-superior-most portion of the mandibular condyle.

Embodiment A9. The method as in any preceding Embodiment, further comprising predicting the mandibular condyle using the scan data input to a machine learning model.

Embodiment A10. The method as in any preceding Embodiment, further comprising generating a jaw model of the patient based on the scan data, the mandibular rotation axis, and the glenoid fossae.

Embodiment A11. The method as in any preceding Embodiment, further comprising predicting a jaw movement of the patient using the jaw model of the patient.

Embodiment A12. The method as in any preceding Embodiment, wherein predicting the jaw movement comprises one or more of a mandibular movement, articular movement, and movement at occlusion of teeth.

Embodiment A13. The method as in any preceding Embodiment, wherein the mandibular movement comprises one or more of a hinge, a protrusion, and a lateral movement.

Embodiment A14. The method as in any preceding Embodiment, wherein the articular movement comprises one or more of a rotation, a translation, a protrusive condylar path, a progressive condylar path, a laterotrusive condylar path, mediotrusive condylar path, a condylar path angle, a Bennett angle, and a Bennett movement.

Embodiment A15. The method as in any preceding Embodiment, wherein the movement at occlusion of teeth comprises one or more of an incisal path, an incisal path angle, an incisal path distance, an occlusal guidance, and an occlusal interference.

Embodiment A16. The method as in any preceding Embodiment, further comprising planning the orthodontic treatment using the jaw model of the patient.

Embodiment A17. The method as in any preceding Embodiment, further comprising generating a plurality of aligner trays with tooth-receiving cavities, each aligner tray corresponding to a respective tooth arrangement.

Embodiment B1. A system for orthodontic treatment planning for a patient, the system comprising:
at least one memory device configured to receive and store three-dimensional volumetric scan data of the dentition; and
at least one processor configured to:
determine, for use in planning an orthodontic treatment, a mandibular rotation axis and a glenoid fossae of the temporal bone based on a mandibular condyle of the patient using the scan data.

Embodiment B2. The system as in any preceding Embodiment, wherein the at least one processor is configured to:
receive three-dimensional intraoral surface scan data of the dentition; and
overlay the intraoral surface scan data and the volumetric scan data to generate integrated scan data.

Embodiment B2. The system as in any preceding Embodiment, wherein overlaying the intraoral surface scan data and the volumetric scan data comprises registering the intraoral surface scan data with the volumetric scan data.

Embodiment B3. The system as in any preceding Embodiment, wherein the volumetric scan data comprises one or more of X-ray scan data and magnetic resonance imaging scan data.

Embodiment B4. The system as in any preceding Embodiment, wherein the volumetric scan data corresponds to a cranium and viscerocranium of the patient.

Embodiment B5. The system as in any preceding Embodiment, wherein the intraoral surface scan data comprises optical color scan data.

Embodiment B6. The system as in any preceding Embodiment, wherein the mandibular rotation axis and the glenoid fossae are based on a lateral, medial, superior, and anterior geometry of the mandibular condyle.

Embodiment B7. The system as in any preceding Embodiment, wherein the mandibular rotation axis and the glenoid fossae are based on the anterio-superior-most portion of the mandibular condyle.

Embodiment B8. The system as in any preceding Embodiment, wherein the at least one processor is configured to predict the mandibular condyle using the scan data input to a machine learning model.

Embodiment B9. The system as in any preceding Embodiment, wherein the at least one processor is configured to generate a jaw model of the patient based on the scan data, the mandibular rotation axis, and the glenoid fossae.

Embodiment B10. The system as in any preceding Embodiment, wherein the at least one processor is configured to predict a jaw movement of the patient using the jaw model of the patient.

Embodiment B11. The system as in any preceding Embodiment, wherein the at least one processor is configured to predict the jaw movement comprises one or more of a mandibular movement, articular movement, and movement at occlusion of teeth.

Embodiment B12. The system as in any preceding Embodiment, wherein the mandibular movement comprises one or more of a hinge, a protrusion, and a lateral movement.

Embodiment B13. The system as in any preceding Embodiment, wherein the articular movement comprises one or more of a rotation, a translation, a protrusive condylar path, a progressive condylar path, a laturotrusive condylar path, mediotrusive condylar path, a condylar path angle, a Bennett angle, and a Bennett movement.

Embodiment B14. The system as in any preceding Embodiment, wherein the movement at occlusion of teeth comprises one or more of an incisal path, an incisal path angle, an incisal path distance, an occlusal guidance, and an occlusal interference.

Embodiment B15. The system as in any preceding Embodiment, further comprising planning the orthodontic treatment using the jaw model of the patient.

Embodiment B16. The system as in any preceding Embodiment, further comprising generating a plurality of aligner trays with tooth-receiving cavities, each aligner tray corresponding to a respective tooth arrangement.

Embodiment B17. The system as in any preceding Embodiment, further comprising a display configured to display the jaw model of the patient and a user interface for navigating the jaw model.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A method of orthodontic treatment planning for a patient, the method comprising:
receiving three-dimensional volumetric scan data of a dentition of the patient; and
determining, for use in planning an orthodontic treatment, a mandibular rotation axis and a glenoid fossae of the temporal bone based on a mandibular condyle of the patient using the scan data.

2. The method of claim 1, further comprising:
receiving three-dimensional intraoral surface scan data of the dentition; and
overlaying the intraoral surface scan data and the volumetric scan data to generate integrated scan data.

3. The method of claim 2, wherein overlaying the intraoral surface scan data and the volumetric scan data comprises registering the intraoral surface scan data with the volumetric scan data.

4. The method of claim 1, wherein the volumetric scan data comprises one or more of X-ray scan data and magnetic resonance imaging scan data.

5. The method of claim 1, wherein the volumetric scan data corresponds to a cranium and viscerocranium of the patient.

6. The method of claim 1, wherein the intraoral surface scan data comprises optical color scan data.

7. The method of claim 1, wherein the mandibular rotation axis and the glenoid fossae are based on a lateral, medial, superior, and anterior geometry of the mandibular condyle.

8. The method of claim 1, wherein the mandibular rotation axis and the glenoid fossae are based on the anterio-superior-most portion of the mandibular condyle.

9. The method of claim 1, further comprising predicting the mandibular condyle using the scan data input to a machine learning model.

10. The method of claim 1, further comprising generating a jaw model of the patient based on the scan data, the mandibular rotation axis, and the glenoid fossae.

11. The method of claim 10, further comprising predicting a jaw movement of the patient using the jaw model of the patient.

12. The method of claim 11, wherein predicting the jaw movement comprises one or more of a mandibular movement, articular movement, and movement at occlusion of teeth.

13. The method of claim 12, wherein the mandibular movement comprises one or more of a hinge, a protrusion, and a lateral movement.

14. The method of claim 12, wherein the articular movement comprises one or more of a rotation, a translation, a protrusive condylar path, a progressive condylar path, a laterotrusive condylar path, mediotrusive condylar path, a condylar path angle, a Bennett angle, and a Bennett movement.

15. The method of claim 12, wherein the movement at occlusion of teeth comprises one or more of an incisal path, an incisal path angle, an incisal path distance, an occlusal guidance, and an occlusal interference.

16. The method of claim 10, further comprising planning the orthodontic treatment using the jaw model of the patient.

17. The method of claim 1, further comprising generating a plurality of aligner trays with tooth-receiving cavities, each aligner tray corresponding to a respective tooth arrangement.

18. A system for orthodontic treatment planning for a patient, the system comprising:
at least one memory device configured to receive and store three-dimensional volumetric scan data of the dentition; and
at least one processor configured to:
determine, for use in planning an orthodontic treatment, a mandibular rotation axis and a glenoid fossae of the temporal bone based on a mandibular condyle of the patient using the scan data.

19. The system of claim 18, wherein the at least one processor is configured to:
receive three-dimensional intraoral surface scan data of the dentition; and
overlay the intraoral surface scan data and the volumetric scan data to generate integrated scan data.

20. The system of claim 19, wherein overlaying the intraoral surface scan data and the volumetric scan data comprises registering the intraoral surface scan data with the volumetric scan data.

21. The system of claim 18, wherein the volumetric scan data comprises one or more of X-ray scan data and magnetic resonance imaging scan data.

22. The system of claim 18, wherein the volumetric scan data corresponds to a cranium and viscerocranium of the patient.

23. The system of claim 18, wherein the intraoral surface scan data comprises optical color scan data.

24. The system of claim 18, wherein the mandibular rotation axis and the glenoid fossae are based on a lateral, medial, superior, and anterior geometry of the mandibular condyle.

25. The system of claim 18, wherein the mandibular rotation axis and the glenoid fossae are based on the anterio-superior-most portion of the mandibular condyle.

26. The system of claim 18, wherein the at least one processor is configured to predict the mandibular condyle using the scan data input to a machine learning model.

27. The system of claim 18, wherein the at least one processor is configured to generate a jaw model of the patient based on the scan data, the mandibular rotation axis, and the glenoid fossae.

28. The system of claim 27, wherein the at least one processor is configured to predict a jaw movement of the patient using the jaw model of the patient.

29. The system of claim 28, wherein the at least one processor is configured to predict the jaw movement comprises one or more of a mandibular movement, articular movement, and movement at occlusion of teeth.

30. The system of claim 29, wherein the mandibular movement comprises one or more of a hinge, a protrusion, and a lateral movement.

31. The system of claim 29, wherein the articular movement comprises one or more of a rotation, a translation, a protrusive condylar path, a progressive condylar path, a laturotrusive condylar path, mediotrusive condylar path, a condylar path angle, a Bennett angle, and a Bennett movement.

32. The system of claim 29, wherein the movement at occlusion of teeth comprises one or more of an incisal path, an incisal path angle, an incisal path distance, an occlusal guidance, and an occlusal interference.

33. The system of claim 27, further comprising planning the orthodontic treatment using the jaw model of the patient.

34. The system of claim 18, further comprising generating a plurality of aligner trays with tooth-receiving cavities, each aligner tray corresponding to a respective tooth arrangement.

35. The system of claim 18, further comprising a display configured to display the jaw model of the patient and a user interface for navigating the jaw model.
